(19) United States
(12) Patent Application Publication
(10) Pub. No.: US 2025/0308675 A1
(43) Pub. Date: Oct. 2, 2025

(54) OCT IMAGE PROCESSING PROGRAM, OCT IMAGE PROCESSING DEVICE, AND OCT IMAGE PROCESSING METHOD

(71) Applicant: NIDEK CO.,LTD., Gamagori-shi (JP)

(72) Inventors: Ryosuke SHIBA, Gamagori-shi (JP); Tetsuya KANO, Gamagori-shi (JP)

(21) Appl. No.: 19/090,424

(22) Filed: Mar. 26, 2025

(30) Foreign Application Priority Data
Mar. 29, 2024 (JP) 2024-055460

(51) Int. Cl. G16H 30/40; G06T 5/60

(52) U.S. Cl. CPC G16H 30/40; G06T 5/60; G06T 2207/10101; G06T 2207/20081; G06T 2207/20084

(57) ABSTRACT

An OCT image processing program causes an OCT image processing device to perform: an image acquisition step of acquiring a shallow layer image and a deep layer image which are generated based on the motion contrast data, wherein the shallow layer image is an image of a shallow region of the living tissue, and the deep layer image is an image of a deep region deeper than the shallow region; a correction weight calculation step of calculating a correction weight for correcting the deep layer image such that a correlation between the shallow layer image and the deep layer image is reduced; and an image correction step of correcting the deep layer image according to the calculated correction weight. At the correction weight calculation step, the correction weight is calculated using a robust estimation method.

### CROSS REFERENCE TO RELATED APPLICATION

This application is based on, and claims, the benefit of priority from Japanese Patent Application No. 2024-055460 on Mar. 29, 2024. The entire disclosure of the above applications is incorporated herein by reference.

### TECHNICAL FIELD

The present disclosure relates to an OCT image processing program, an OCT image processing device, and an OCT image processing device that are used for processing OCT images of living tissue acquired based on optical coherence tomography (OCT) principles.

### BACKGROUND

Conventionally, techniques for acquiring motion contrast data of living tissue (e.g., the fundus of an eye) based on OCT principles have been proposed. Motion contrast data is obtained by processing multiple OCT signals acquired at the same position on living tissue over different time points. Motion contrast data reflects biological movements (e.g., blood flow within tissue vessels). Data indicating vascular positions in living tissue (angiography data) is an example of motion contrast data.

By processing motion contrast data, it is possible to acquire images of multiple regions at different depths within the living tissue. Here, a scenario is assumed where an image (shallow layer image) of a first depth region (shallow layer) and an image (deep layer image) of a second depth region (deep region) deeper than the first depth region (deep layer image) are acquired based on motion contrast data acquired for the same living tissue. In this case, signals originating from motion in the shallow layer (e.g., blood flow) may appear as artifacts (hereinafter referred to as “projection artifacts”) in the deep layer image.

The ophthalmic image processing device described in Patent Document 1 (JP2019-150405) sets a weight to reduce the correlation between shallow and deep layer images and corrects the deep layer image based on this weight. Ideally, the correlation between shallow and deep layer images should be small because their vascular structures differ. However, stronger projection artifacts increase this correlation. In Patent Document 1, the effects of projection artifacts are reduced by correcting the shallow layer image so that the correlation between the shallow layer image and the deep layer image is reduced.

### SUMMARY

In the technology of Patent Document 1, the weight for reducing the correlation between shallow and deep layer images is set as a correction weight for the deep layer image. However, outliers present in at least one of the shallow or deep layer images can prevent appropriate weight calculation. As a result, projection artifacts may not be sufficiently reduced.

One objective of the present disclosure is to provide an OCT image processing program, an OCT image processing device, and an OCT image processing method that are capable of more effectively reducing the impact of artifacts in deep layer images.

In a first aspect of the present disclosure, an OCT image processing program is executed by an OCT image processing device that processes an image acquired by an OCT device that is configured to generate motion contrast data by processing a plurality of OCT signals acquired from a same position on a living tissue at different times. The OCT image processing program, when executed by a control unit of the OCT image processing device, causes the OCT image processing device to perform: an image acquisition step of acquiring a shallow image and a deep image which are generated based on the motion contrast data, wherein the shallow image is an image of a shallow region of the living tissue, and the deep image is an image of a deep region deeper than the shallow region; a correction weight calculation step of calculating a correction weight for correcting the deep image such that a correlation between the shallow image and the deep image is reduced; and an image correction step of correcting the deep layer image according to the calculated correction weight. At the correction weight calculation step, the correction weight is calculated using a robust estimation method.

In a second aspect of the present disclosure, an OCT image processing device processes an image acquired by an OCT device that is configured to generate motion contrast data by processing a plurality of OCT signals acquired from a same position on a living tissue at different times. The OCT image processing device includes a control unit configured to perform: an image acquisition step of acquiring a shallow image and a deep image which are generated based on the motion contrast data, wherein the shallow image is an image of a shallow region of the living tissue, and the deep image is an image of a deep region deeper than the shallow region; a correction weight calculation step of calculating a correction weight for correcting the deep image such that a correlation between the shallow image and the deep image is reduced; and an image correction step of correcting the deep layer image according to the calculated correction weight. At the correction weight calculation step, the correction weight is calculated using a robust estimation method.

In a third aspect of the present disclosure, an OCT image processing method is a method for processing an image acquired by an OCT device that is configured to generate motion contrast data by processing a plurality of OCT signals acquired from a same position on a living tissue at different times. The OCT image processing method includes: an image acquisition step of acquiring a shallow image and a deep image which are generated based on the motion contrast data, wherein the shallow image is an image of a shallow region of the living tissue, and the deep image is an image of a deep region deeper than the shallow region; a correction weight calculation step of calculating a correction weight for correcting the deep image such that a correlation between the shallow image and the deep image is reduced; and an image correction step of correcting the deep layer image according to the calculated correction weight. At the correction weight calculation step, the correction weight is calculated using a robust estimation method.

The OCT image processing program, the OCT image processing device, and the OCT image processing method in the present disclosure enable more effective reduction of artifacts in deep layer images.

### BRIEF DESCRIPTION OF DRAWINGS

FIG. 1 is a block diagram showing a schematic configuration of an OCT device (OCT image processing device) 1.

FIG. 2 is a flowchart of OCT image processing executed by the OCT device.

FIG. 3 is an explanatory diagram illustrating an example method for acquiring interference signals from a two-dimensional measurement area 55 on a living tissue.

FIG. 4 is an explanatory diagram illustrating a method for acquiring multiple frames of interference signals from the same scan line at different times.

FIG. 5 is a diagram showing an example result of layer/boundary detection from a B-scan image.

FIG. 6 is a diagram showing examples of a shallow layer image 60 and a deep layer image 70 generated from the same motion contrast data.

FIG. 7 is a diagram showing the shallow layer image 60 and deep layer image 70 divided into multiple local regions LA.

FIG. 8 is a diagram showing a co-occurrence histogram of the shallow and deep layer images.

FIG. 9 is an explanatory diagram illustrating the relationship between a co-occurrence histogram in FIG. 8 and the slopes of two correction weights w and w′.

FIG. 10 is a diagram showing examples of corrected images 80 and 80′ obtained by correcting the deep layer image 70 using the two correction weights w and w′.

### DETAILED DESCRIPTION

### Overview

The OCT image processing device exemplified in the present disclosure processes images acquired by an OCT device. An OCT image processing program is executed by a control unit of the OCT image processing device. The OCT device generates motion contrast data by processing multiple OCT signals acquired from the same position on living tissue at different times. The control unit executes an image acquisition step, a correction weight calculation step, and an image correction step. In the image acquisition step, the control unit acquires a shallow layer image and a deep layer image generated based on motion contrast data acquired for the same living tissue. A shallow layer image is an image of a shallow region (an area shallower than a deep region) of living tissue. A deep layer image is an image of a deep region of living tissue that is deeper than a shallow region. Here, projection artifacts are considered to arise when vascular structures from the shallow layer appear in the deep layer image. Thus, in regions where projection artifacts are present, the same features (e.g., blood vessels) are likely to appear in both the shallow and deep layer images. To remove projection artifacts, a process is performed to eliminate features common to both the shallow and deep layer images. This process is achieved by reducing the correlation between the shallow and deep layer images. In the correction weight calculation step, the control unit calculates a correction weight to correct the deep layer image such that the correlation between the shallow and deep layer images is minimized (e.g., the correlation is reduced as much as possible). In the image correction step, the control unit corrects the deep layer image according to the calculated correction weight. In the correction weight calculation step, the control unit calculates the correction weight using a robust estimation method.

As a method to decorrelate the shallow and deep layer images, principal component analysis (PCA) can be considered. As mentioned earlier, projection artifacts appear in both the shallow and deep layer images, so they are likely to constitute the first principal component when PCA is applied to these images. Therefore, by visualizing the second principal component scores, a deep layer image free of projection artifacts can theoretically be obtained.

However, while the PCA-based method produces images where projection artifacts appear reduced, the resulting images contain information from both the shallow and deep layers. This makes it unsuitable as a proper processing method for observing the deep layer independently.

Instead of using PCA-transformed principal component scores, the slope of the first principal component is used to correct the deep layer image. The slope is defined as the correction weight w, which is multiplied by the shallow layer image E shallow and subtracted from the deep layer image E deep to obtain the corrected deep layer image E′_{deep}. This process is expressed by the following equation:

$${E}_{deep}^{′} = {E}_{deep} - w \times {E}_{shawllow}$$

The above equation is expected to yield a deep layer image free of projection artifacts. However, the slope derived from PCA generally differs from the slope of the line that minimizes residuals from the shallow to the deep layer image. In order to minimize residuals from a shallow layer image to a deep layer image, a line converting shallow layer image pixels to deep layer image pixels can be calculated using least squares regression, and its slope can be used as the correction weight w in the above equation.

However, the least squares method assumes that only projection artifacts commonly shown in shallow layer images and deep layer images exist. In reality, numerous outliers exist, such as vessels appearing only in the deep layer images, vessels appearing only in the shallow layer images, and noises. In the aforementioned processing, the presence of numerous outliers degrades the accuracy of weight calculation via the least squares method, resulting in cases where projection artifacts are not adequately removed.

In contrast to this, the OCT image processing device of the present disclosure uses a robust estimation method to reduce the effects of outliers and calculate correction weights w. As a result, the impact of artifacts in the deep layer image is more effectively reduced.

In the correction weight calculation step, the correction weight may be calculated using a robust estimation method that incorporates a shallow layer weight derived from the shallow layer image. As previously described, projection artifacts appearing in the deep layer image are considered to arise from the overlay of the shallow layer image onto the deep layer image. Thus, by calculating the correction weight using a robust estimation method that employs the weight of the shallow layer image itself (shallow layer image weight), the correction weight is derived while more effectively excluding the influence of outliers. As a result, the impact of artifacts in the deep layer image is more effectively reduced.

As a robust estimation method, weighted regression analysis (e.g., weighted least squares) may be used to calculate the correction weight. Weighted regression assigns lower weights to less reliable data (likely outliers), suppressing their impact on the regression results. Thus, weighted regression ensures that the correction weight is calculated with minimal influence from outliers.

However, the specific method for calculating the correction weight can also be modified. Other robust methods such as RANSAC (Random Sample Consensus), M-estimation, or Least Median of Squares (LMedS) may be used to calculate the correction weight.

In the correction weight calculation step, if “m” is the shallow layer weight, “x” is the shallow layer brightness, and “y” is the deep layer brightness, the correction weight w may be calculated using Equation 1: This equation further reduces the influence of outliers.

$$w = \frac{\sum m \sum mxy - \sum mx \sum my}{\sum m \sum {mx}^{2} - {( \sum mx )}^{2}} \qquad [ Equation 1 ]$$

For example, the shallow layer image brightness can be normalized to a range of 0 to 1, and its square can be used as the shallow layer image weight m. In this case, the background brightness becomes nearly zero, making it easier to retain information from strong signals.

In the correction weight calculation step, the control unit may perform the calculation per local region. In the image correction step, the deep layer image may be corrected per local region based on the calculated weights. In this case, if the influence of projection artifacts in a certain local area is large, the correlation between the shallow layer image and the deep layer image increases, so the correction weight increases. On the other hand, if the influence of projection artifacts in a certain local area is small, the correlation between the shallow layer image and the deep layer image decreases, so the correction weight becomes smaller. Therefore, by performing the processing per local region, the influence of artifacts generated in the deep layer image is more effectively reduced.

When calculating the correction weight using the shallow layer image weight, the control unit may perform both the calculation of the shallow layer image weight from the shallow layer image and the calculation of the correction weight using the shallow layer image weight per local region. In this case, since the shallow layer image weight is also calculated per local region, the influence of outliers is appropriately reduced according to the local region.

### Embodiment

Hereinafter, one of exemplary embodiments according to the present disclosure will be described. As an example, the OCT device 1 in this embodiment is capable of processing OCT signals obtained from the fundus tissue of the subject eye E. However, at least part of the techniques exemplified in the present disclosure can also be applied when processing OCT signals from living tissues other than the fundus of the subject eye E or living tissues other than the subject eye E (e.g., skin, digestive organs, brain, or blood vessels, including cardiovascular vessels). OCT data refers to data acquired based on the principles of optical coherence tomography (OCT).

In this embodiment, the OCT device 1 itself functions as an OCT image processing device by executing the various processing operations described later. However, devices capable of functioning as OCT image processing devices are not limited to the OCT device 1. For example, a PC or another device capable of acquiring OCT signals or OCT images generated by OCT device 1 may function as an OCT image processing device.

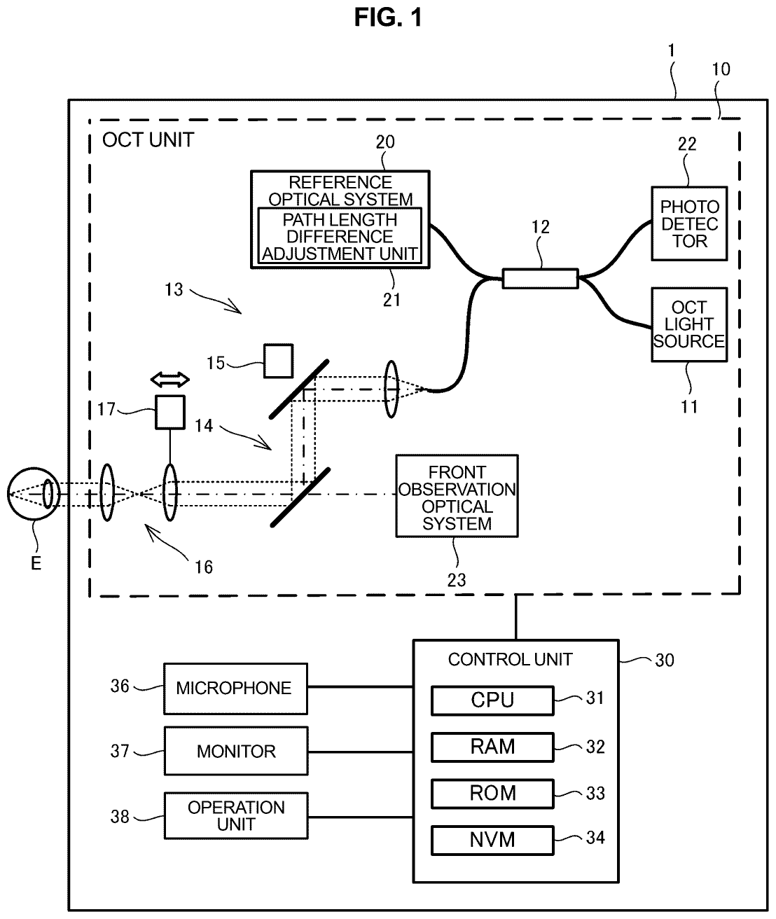

Referring to FIG. 1, the schematic configuration of the OCT device (i.e., an OCT image processing device) 1 in the present embodiment is described. The OCT device 1 comprises an OCT unit 10 and a control unit 30. The OCT unit 10 comprises an OCT light source 11, a coupler (optical splitter) 12, a measurement optical system 13, a reference optical system 20, a photodetector 22, and a front observation optical system 23.

The OCT light source 11 emits light (OCT light) in order to acquire image data. The coupler 12 divides the OCT light emitted from the OCT light source 11 into a measurement beam and a reference beam. Further, the coupler 12 of the present embodiment combines the measurement beam reflected by the living tissue (the fundus of the subject eye E in the present embodiment) and the reference beam generated by the reference optical system 20 to have the measurement beam and the reference beam interfere with each other. That is, the coupler 12 of the present embodiment also serves as both a branching optical element that branches the OCT light into the measurement beam and the reference beam and a combined wave optical element that combines the reflected light of the measurement beam and the reference beam. The configuration of at least one of the branching optical element and the combined wave optical element may be changed. For example, an element other than the coupler (for example, a circulator, a beam splitter, or the like) may be used.

The measurement optical system 13 directs the measurement beam split by coupler 12 to the subject eye and routes the reflected measurement beam back to coupler 12. The measurement optical system 13 comprises a scanning unit 14, an illumination optical system 16, and a focus adjustment unit 17. The scanning unit 14 is configured to deflect the measurement beam in two-dimensional directions perpendicular to the optical axis when driven by the driving unit 15. In the present embodiment, two galvanometer mirrors capable of deflecting the measurement beam in different directions from each other are used as the scanning unit 14. However, another device (for example, at least one of a polygon mirror, resonant scanner, acousto-optic element, or the like) that deflects light may be used as the scanning unit 14. The illumination optical system 16 is positioned downstream of the scanner unit 14 in the optical path (i.e., on the subject side) and irradiates the measurement beam onto the subject's tissue. The focus adjustment unit 17 adjusts the focus of the measurement beam by moving optical components (e.g., a lens) provided in the illumination optical system 16 along the optical axis of the measurement beam.

The reference optical system 20 generates reference beam and returns it to the coupler 12. In the present embodiment, the reference optical system 20 generates reference beam by reflecting the reference beam split by the coupler 12 using a reflective optical system (e.g., a reference mirror). However, the configuration of the reference optical system 20 can be modified. For example, the reference optical system 20 may transmit the light incident from the coupler 12 without reflection and return it to the coupler 12. The reference optical system 20 includes a path length difference adjustment unit 21 that changes the path length difference between the measurement and reference beams. In the present embodiment, the path length difference is changed by moving the reference mirror along the optical axis. Additionally, the configuration for changing the path length difference may also be placed in the measurement optical system 13.

The photodetector 22 detects an interference signal by receiving interference light generated by the coupler 12 from the measurement and reference beams. In the present embodiment, the principle of Fourier-domain OCT is adopted. In Fourier-domain OCT, the spectral intensity (spectral interference signal) of the interference light is detected by the photodetector 22, and complex OCT signals are obtained by performing a Fourier transform on the spectral intensity data. As an example of Fourier domain OCT, such as Spectral-domain OCT (SD-OCT) and Swept-source OCT (SS-OCT), and the like can be used. Further, for example, Time-domain OCT (TD-OCT) and the like can also be used.

In this embodiment, SD-OCT is adopted. In SD-OCT, for example, a low-coherence light source (broadband light source) is used as the OCT light source 11, and a spectroscopic optical system (spectrometer) that spectrally separates the interference light into its frequency components (wavelength components) is provided near the photodetector 22 in the optical path of the interference light. In SS-OCT, for example, a wavelength-swept light source (tunable light source) that rapidly changes its emission wavelength over time is used as the OCT light source 11. In this case, the OCT light source 11 may include a light source, a fiber ring resonator, and a wavelength selection filter. The wavelength selection filter may be, for example, a filter combining a diffraction grating and a polygon mirror, or a filter using a Fabry-Pérot etalon.

In the present embodiment, three-dimensional OCT data (e.g., three-dimensional cross-sectional images) are acquired by scanning the measurement beam spot in a two-dimensional measurement area using the scanning unit 14. However, the acquisition principle for three-dimensional OCT data can be modified. For example, three-dimensional OCT data may be obtained using the principle of Line-Field OCT (LF-OCT). In LF-OCT, the measurement beam is simultaneously projected onto a line-shaped illumination area on the tissue, and the interference light between the reflected measurement beam and the reference beam is detected by a one-dimensional photodetector (e.g., line sensor) or a two-dimensional photodetector. Three-dimensional OCT data are acquired by scanning the measurement beam in a direction intersecting the illumination line within the two-dimensional measurement area. Alternatively, three-dimensional OCT data may be obtained using the principle of Full-Field OCT (FF-OCT). In FF-OCT, measurement beam is projected onto a two-dimensional measurement area on the tissue, and interference light between reflected measurement beam and reference beam is detected by a two-dimensional photodetector. In this case, the OCT device 1 may operate without the scanning unit 14.

The front observation optical system 23 is provided to capture real-time front observation images of the subject's living tissue (in the present embodiment, the eye fundus of the subject eye E). In the present embodiment, the front observation image refers to a two-dimensional image viewed from the front direction (along the optical axis of the OCT measurement beam). In the present embodiment, a scanning laser ophthalmoscope (SLO) is adopted as the front observation optical system 23. However, the front observation optical system 23 may also adopt other configurations (e.g., an infrared camera that irradiates the two-dimensional imaging area with infrared light to capture front images).

The OCT device 1 can acquire (generate) an en face image, which is a two-dimensional front image viewed from the front direction (along the optical axis of the measurement beam) based on the acquired three-dimensional OCT data. When generating en face images in real-time, the obtained en face images can also be used as the aforementioned front observation images. In this case, the front observation optical system 23 may be omitted. The en face image data may be, for example, accumulated intensity data where brightness values are summed along the depth direction (Z-direction) at each XY position, accumulated spectral data values at each XY position, brightness data at each XY position within a specific depth direction, or brightness data at each XY position in a specific retinal layer (e.g., retinal surface layer). The OCT device 1 in the present embodiment can also generate en face images from motion contrast data. Motion contrast data is obtained by processing multiple OCT signals acquired at the same position on living tissue over different time points. Motion contrast data reflects biological movements (e.g., blood flow within tissue vessels). In the present embodiment, en face images of specific layers generated from motion contrast data can produce angiography images (vascular images) indicating vascular positions within those layers. Additionally, the OCT device 1 can generate multiple OCT images (e.g., two-dimensional en face images) from motion contrast data acquired from the same living tissue across different depth regions.

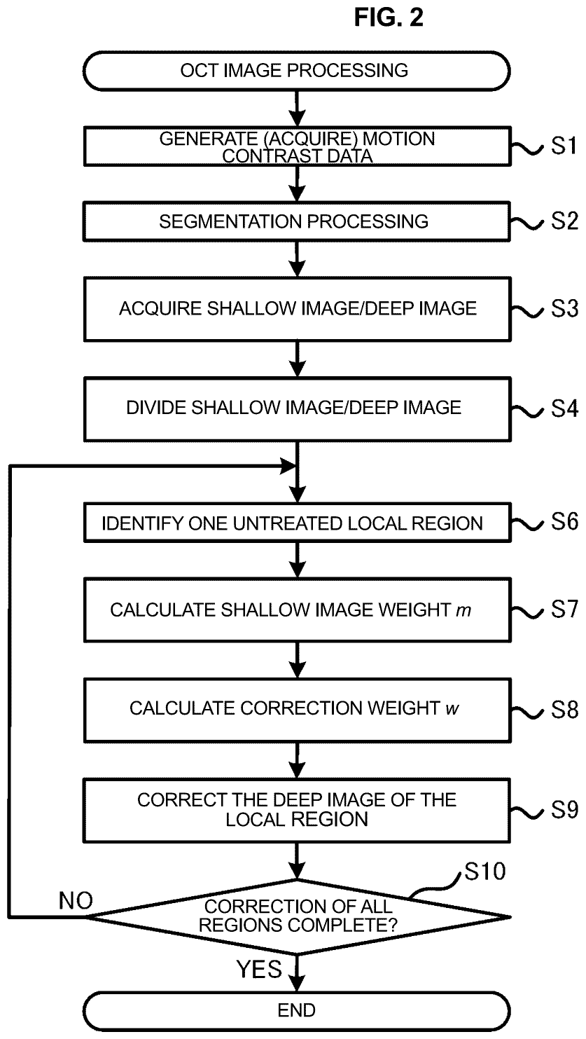

The control unit 30 governs various controls of the OCT device 1. The control unit 30 comprises a CPU 31, RAM 32, ROM 33, and non-volatile memory (NVM) 34. The CPU 31 is a controller that performs various control operations. The RAM 32 temporarily stores various information. The ROM 33 stores programs to be executed by the CPU 31, various initial values, and the like. The NVM 34 is a non-transitory storage medium that retains storage contents even when a power supply is interrupted. The OCT image processing program for executing the OCT image processing (refer to FIG. 2) may also be stored in the NVM 34.

The control unit 30 is connected to a microphone 36, a monitor 37, and an operation unit 38. The microphone 36 is used for audio input. The monitor 37 is an example of a display unit for showing various images The operation unit 38 is operated by the user to input various operational instructions into the OCT device 1. For example, various devices such as a mouse, keyboard, touch panel, and foot switch may be used as the operation unit 38. Additionally, operational instructions can be input into the OCT device 1 via audio input through the microphone 36. In this case, the CPU 31 may perform speech recognition processing on the input audio to determine the type of operational instruction.

In the present embodiment, the OCT device 1 is exemplified as a single-unit system with the OCT part 10 and the control unit 30 integrated into one chassis. However, the OCT device 1 may also consist of multiple units with different chassis. For example, the OCT device 1 may comprise an optical device incorporating the OCT part 10 and a PC connected via a wired or wireless connection to the optical device. In this case, the control sections of the optical device and the PC may collectively function as the control unit 30 of the OCT device 1.

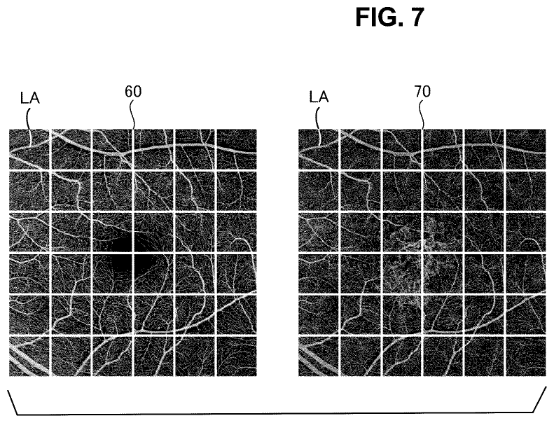

Referring to FIG. 2 to FIG. 10, the OCT image processing device (in the present embodiment, the OCT device 1) performs OCT image processing. In the OCT image processing, shallow layer images 60 and deep layer images 70 (see FIGS. 6 and 7) are obtained based on the same motion contrast data. The deep layer image 70 is corrected to reduce the influence of projection artifacts caused by signals of the shallow layer image 60 signals. The CPU 31 of the OCT device 1 executes an OCT image processing program stored in the NVM 34 according to the OCT image processing shown in FIG. 2.

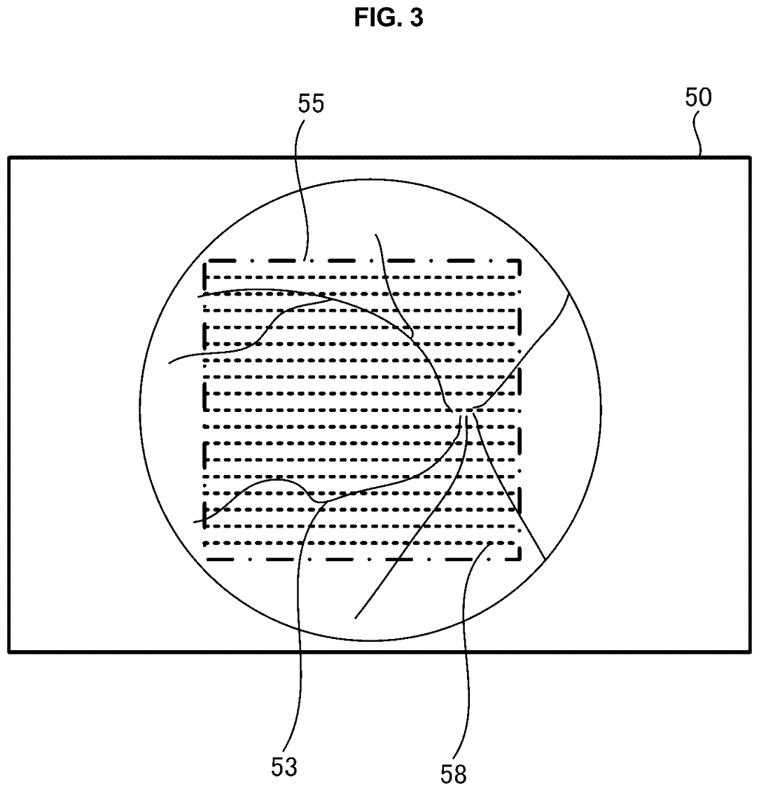

The CPU 31 generates (obtains) motion contrast data of the living tissue of the subject (in the present embodiment, the fundus of subject eye E) (S1). First, the CPU 31 controls the front observation optical system 23 to start capturing a two-dimensional front image of the living tissue targeted for interference signal acquisition. In the example shown in FIG. 3, the two-dimensional front image 50 includes retinal blood vessels 53, etc. The two-dimensional front image 50 is repeatedly captured and displayed as a video on the monitor 37.

The CPU 31 starts acquiring interference signals upon generation of a trigger signal for initiating interference signal acquisition. In the present embodiment, the CPU 31 controls the drive unit 15 to drive the scanning unit 14, thereby scanning the measurement spot across the two-dimensional measurement area 55 to acquire interference signals for the measurement area 55. For example, as shown in FIG. 3, the present embodiment sets straight scan lines 58 at equal intervals within the measurement area 55 and scans the measurement spot along each scan line 58 to acquire interference signals for the two-dimensional measurement area 55.

More precisely, the CPU 31 acquires at least 2 frames of interference signals at the same position (in the example shown in FIG. 3, the same scan line 58) at different times. In the example shown in FIG. 3, the CPU 31 first scans the measurement beam along the first scan line 58 among multiple scan lines 58, thereby acquiring interference signals detected by the photodetector 22. Below, the direction along which the scan line 58 extends is defined as the X-direction. Scanning with the measurement beam once in the X-direction along each scan line 58 is referred to as a “B-scan.” The two-dimensional image generated by a B-scan is called a “B-scan image.” Among the B-scan images, multiple pixel columns extending along the direction of the measurement beam's optical axis are each referred to as an “A-scan image.” Below, 1 frame of interference signal is described as the interference signal acquired by a single B-scan. The Z-direction is defined as the direction along the measurement beam's optical axis. The Y-direction is the direction intersecting both the X-direction and Z-direction (in the present embodiment, the direction perpendicular to both).”

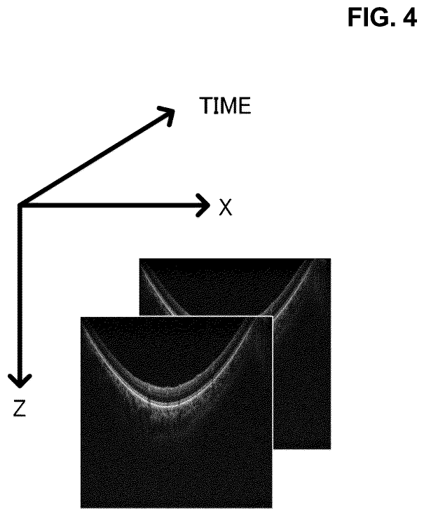

Once the first B-scan on the first scan line 58 is completed, the CPU 31 executes a second B-scan on the first scan line 58 to acquire the second frame of interference signals. As a result, as shown in FIG. 4, two frames of interference signals are obtained from the first scan line 58 at different times. Additionally, the CPU 31 can acquire 3 or more frames of interference signals from the same position (e.g., the same scan line 58). Furthermore, if multiple frames of interference signals can be obtained by scanning the measurement beam once along the scan line 58 (for example, by simultaneously scanning two measurement beams whose optical axes are shifted at predetermined intervals), then it is not necessary to scan the measurement beam multiple times along the same scan line 58.

After completing the acquisition of multiple frames of interference signals from the first scan line 58, the CPU 31 moves the B-scan execution position parallel to the Y-direction and executes the acquisition process for multiple frames of interference signals from the second scan line 58. By executing the above process for each of the multiple scan lines 58, interference signals for the two-dimensional measurement area 55 are acquired. Additionally, the direction of the first B-scan and the second B-scan on the same scan line 58 can be reversed, or multiple B-scans can be repeated in the same direction.

The CPU 31 performs a Fourier transform on the acquired interference signals to obtain complex OCT signals. The CPU 31 executes image registration (alignment) of the positions of multiple complex OCT signals acquired at the same living tissue location at different times and corrects the phase differences between them. Subsequently, the CPU 31 generates (obtains) motion contrast data based on the changes in the multiple complex OCT signals.

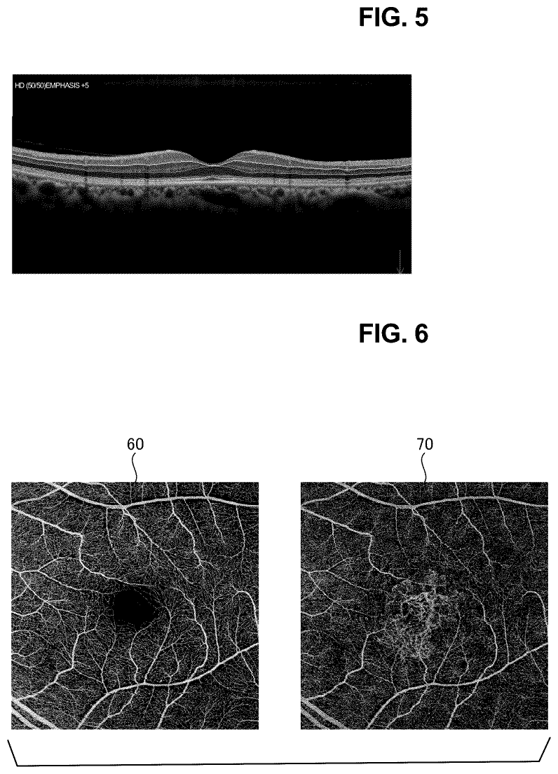

Subsequently, the CPU 31 executes segmentation processing to detect at least one of the layers or boundaries of living tissue from at least part of multiple B-scan images generated by multiple complex OCT signals (S2). FIG. 5 shows an example of a result where multiple layers and boundaries were detected from B-scan images. For example, in the present embodiment, a mathematical model trained using a machine learning algorithm is employed. The mathematical model is pre-trained using multiple training datasets including B-scan images to output layer/boundary detection results for input B-scan images. The CPU 31 inputs B-scan images into the mathematical model to obtain layer/boundary detection results. However, other methods (e.g., publicly known image processing methods) may also be used for the segmentation processing. The S2 processing is executed for each of the multiple B-scan images acquired for each of the multiple scan lines 58.

The CPU 31 generates (obtains) shallow layer image 60 and deep layer image 70 of the same living tissue based on the motion contrast data acquired in S1 and the results of the segmentation processing executed in S2 (S3). shallow layer image 60 refers to the image of the first depth region (shallow region). deep layer image 70 refers to the image of the second depth region (deep region), which is deeper than the first depth region.

As shown in FIG. 6, in the present embodiment, en face images (two-dimensional frontal images obtained by viewing the tissue along the optical axis direction of the OCT measurement beam) are acquired as shallow layer image 60 and deep layer image 70. The en face image data may be, for example, accumulated intensity data where brightness values are summed along the depth direction (Z-direction) at each XY position, accumulated spectral data values at each XY position, brightness data at each XY position within a specific depth direction, or brightness data at each XY position in a specific retinal layer (e.g., retinal surface layer). As described earlier, the motion contrast data generated in S1 contains information about tissue movement (e.g., blood flow movement within vessels in living tissue). In the present embodiment, shallow layer image 60 and deep layer image 70 (both en face images) are obtained based on the motion contrast data. Therefore, shallow layer image 60 and deep layer image 70 in the present embodiment are angiography images (vascular images) that indicate the positions of vessels in specific layers.

The CPU 31 divides the image regions of shallow layer image 60 and deep layer image 70 obtained in S3 into multiple local regions LA (S4). In the example shown in FIG. 7, the image regions of shallow layer image 60 and deep layer image 70 are each divided into 6×6 grid-like local regions LA (36 in total). Each local region LA has uniform shape and size. However, the specific method for dividing the image regions into multiple local regions may be altered. shallow layer image 60 and deep layer image 70 are both derived from the same motion contrast data. Therefore, the XY-direction positions of shallow layer image 60 and deep layer image 70 match, allowing the XY-direction regions of both images to be unambiguously identified.

Subsequently, the CPU 31 executes correction processing to reduce the influence of projection artifacts caused by shallow layer image 60 on deep layer image 70 (S6 to S10). Below, the correction processing algorithm for deep layer image 70 in the present embodiment is explained in comparison with conventional correction processing. In reality, the vessels in the shallow region (where shallow layer image 60 is acquired) and the vessels in the deep region (where deep layer image 70 is acquired) are different. Thus, ideally, the correlation between shallow layer image 60 and deep layer image 70 should be small. However, as described earlier, since shallow layer image 60 and deep layer image 70 are both derived from the same motion contrast data, signals originating from tissue movement (e.g., blood flow in vessels as in the present embodiment) in the shallow region appear as projection artifacts in deep layer image 70. Specifically, if the deep layer image 70 acquired in S3 is denoted as ‘E_{deep},’ the shallow layer image 60 acquired in S3 as ‘E_{shallow},’ the projection artifact as ‘p,’ and the true deep layer image (with projection artifacts removed) as ‘E′_{deep},’ then deep layer image 70 ‘E_{deep}’ can be expressed by the following (Equation 2):

$${E}_{deep} = {E}_{deep}^{′} + p \qquad [ Equation 2 ]$$

The projection artifact “p” can be considered as the shallow layer image 60 multiplied by a correction weight “w.” Therefore, the projection artifact “p” can be expressed by the following (Equation 3):

$$p = w \times {E}_{shallow} \qquad [ Equation 3 ]$$

Therefore, if an appropriate correction weight “w” is calculated, the true deep layer image “E′_{deep}” with the projection artifact removed can be obtained using the following (Equation 4):

$${E}_{deep}^{′} = {E}_{deep} - w \times {E}_{shawllow} \qquad [ Equation 4 ]$$

Ideally, the correlation between shallow layer image 60 and deep layer image 70 should be small. Consequently, in the previously mentioned Patent Document 1, a weight that minimizes the correlation between shallow layer image 60 and deep layer image 70 is calculated as the correction weight for correcting deep layer image 70, using the following (Equation 5):

$$w = \frac{Cov ( {E}_{shallow} , {E}_{deep} )}{Var ( {E}_{shallow} )} \qquad [ Equation 5 ]$$

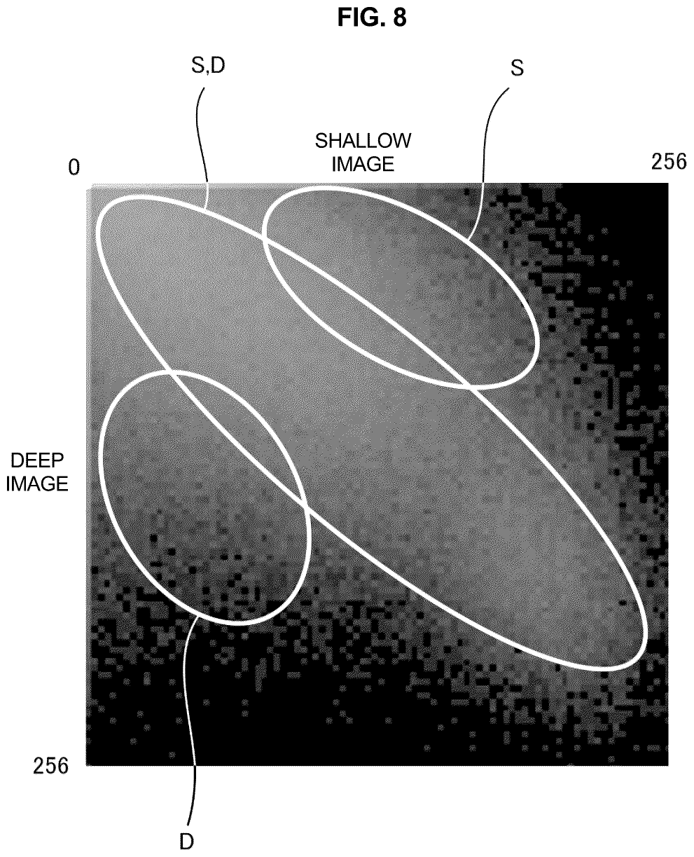

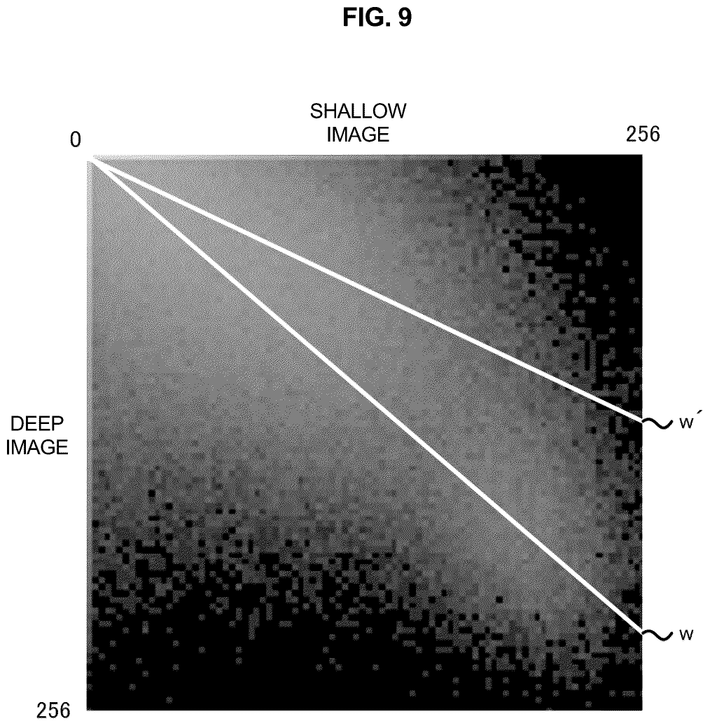

Below, the method for calculating the correction weight “w” in the present embodiment is explained. FIG. 8 and FIG. 9 show examples of co-occurrence histograms (also called “inter-image co-occurrence histograms”) that plot the brightness values of pixels at the same positions in shallow layer image 60 and deep layer image 70. Specifically, the co-occurrence histograms shown in FIG. 8 and FIG. 9 represent the frequency of occurrence of pairs of brightness values for pixels at the same coordinates in shallow layer image 60 and deep layer image 70, determined across the entire image region. In the examples shown in FIG. 8 and FIG. 9, In the examples shown in FIGS. 8 and 9, the brightness of each pixel in the co-occurrence histogram indicates the frequency of occurrence.

In the example shown in FIG. 8, regions S and D in the co-occurrence histogram contain many pixels that are common to both shallow layer image 60 and deep layer image 70. These include areas corresponding to thick vessels in shallow layer image 60 and signals from vessels in shallow layer image 60 that appear as projection artifacts in deep layer image 70. Additionally, region S contains pixels that are only present in shallow layer image 60, primarily representing capillaries and similar structures. Region D contains pixels that are only present in deep layer image 70, primarily representing neovascularization and similar features. The pixel information in regions S and D is likely from areas less influenced by projection artifacts. In other words, the pixel information in regions S and D tends to act as outliers that are unnecessary for calculating the correction weight “w” to reduce the influence of projection artifacts.

Therefore, as shown in FIG. 9, if a correction weight “w” is simply calculated to minimize the correlation between shallow layer image 60 and deep layer image 70, the calculated correction weight “w” is likely to be influenced by the pixel information present in only one of regions S and D, acting as outliers. In the example shown in FIG. 9, the correction weight “w” differs from the ideal correction weight “w” due to the significant influence of the pixel information present only in shallow layer image 60.

In contrast, the OCT device 1 of the present embodiment calculates the correction weight “w” (see FIG. 9) using a robust estimation method that incorporates a shallow layer image weight “m” derived from shallow layer image 60. The projection artifact appearing in deep layer image 70 is considered to be the result of the shallow layer image 60 being superimposed on deep layer image 70. Therefore, by calculating the correction weight “w” using a robust estimation method with the weight of shallow layer image 60 itself (shallow layer image weight “m”), the influence of outliers is more appropriately excluded when determining the correction weight “m.” As a result, the impact of artifacts in deep layer image 70 is more effectively reduced.

As an example, the OCT device 1 of the present embodiment calculates the correction weight using weighted regression analysis (e.g., weighted least squares method) with the shallow layer image weight “m.” Weighted regression assigns lower weights to less reliable data (likely outliers), suppressing their impact on the regression results. Therefore, the use of weighted regression analysis ensures that the correction weight “w” is calculated while more effectively excluding the influence of outliers.

More specifically, in the OCT device 1 of the present embodiment, the correction weight “w” is calculated using the following equation (Equation 1), where “m” is the shallow layer image weight, “x” is the brightness of shallow layer image 60, and “y” is the brightness of deep layer image 70. In this embodiment, the brightness of shallow layer image 60 is normalized so that the minimum brightness is 0 and the maximum brightness is 1. The square of this normalized brightness is used as the shallow layer image weight “m,” which effectively reduces the background brightness to nearly 0 while preserving the information of strong signals.

$$w = \frac{\sum m \sum mxy - \sum mx \sum my}{\sum m \sum {mx}^{2} - {( \sum mx )}^{2}} \qquad [ Equation 1 ]$$

Returning to the explanation of FIG. 2, the details of the correction processing (S6 to S10) for deep layer image 70 in the present embodiment are described. First, the CPU 31 identifies one of the local regions LA divided in S4 that has not yet been processed (S6). The CPU 31 then calculates the shallow layer image weight “m” from the image information of the local region LA identified in S6, within the image region of shallow layer image 60 acquired in S3 (S7).

Next, the CPU 31 calculates the correction weight “w” for correcting the deep layer image 70 of the local region LA identified in S6, using a robust estimation method (in this embodiment, the weighted least squares method, a type of weighted regression analysis) with the shallow layer image weight “m” calculated in S7 (S8). As described earlier, in this embodiment, the correction weight “w” is calculated using Equation 1. The CPU 31 then corrects the image of the local region LA identified in S6 within the deep layer image 70 acquired in S3, based on the correction weight “w” calculated in S8 (S9). If the correction has not been completed for all local regions LA (S10: NO), the process returns to S6, and the correction processing is executed for the next local region LA (S6 to S10). Once the correction is completed for all local regions LA (S10: YES), the process ends.

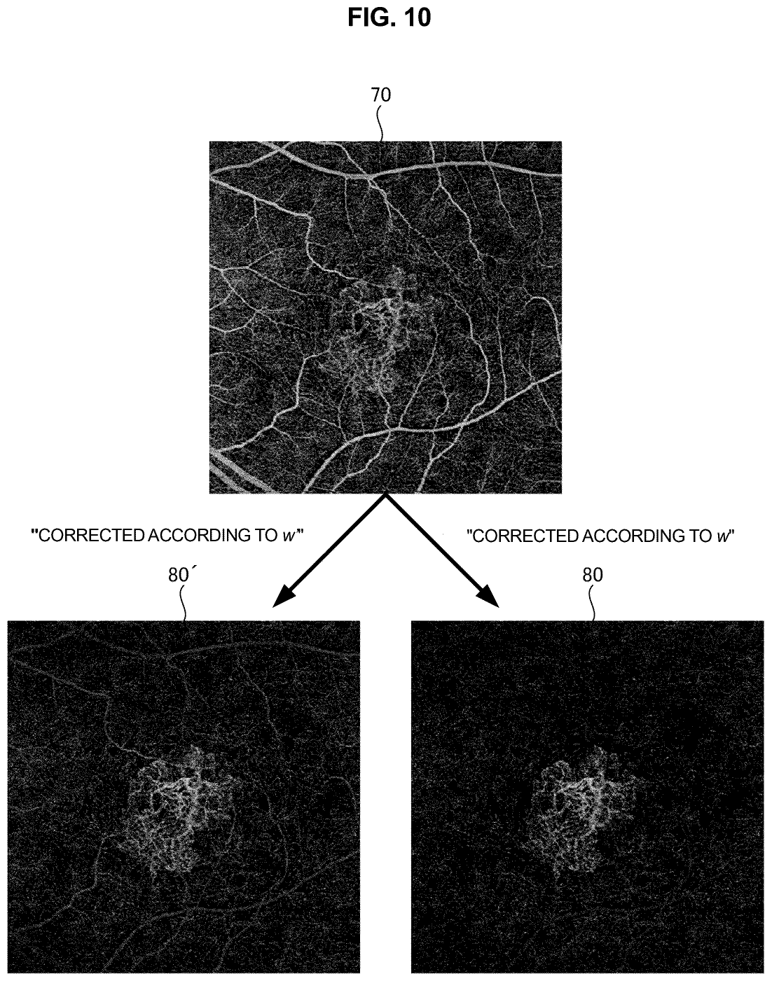

FIG. 10 compares the results of the conventional correction processing (using the correction weight “w” that simply minimizes the correlation between shallow layer image 60 and deep layer image 70) and the correction processing of the present embodiment (using the correction weight “w” derived from a robust estimation method with the shallow layer image weight “m”). Both corrected images 80 and 80′ shown in FIG. 10 are generated by processing the same deep layer image 70 according to their respective correction weights. However, in the corrected image 80′ generated by the conventional correction processing (using the correction weight “w”), signals originating from vessels in shallow layer image 60 still remain as projection artifacts. This is likely due to the influence of outliers, which prevents the correction weight “w” from being calculated appropriately. In contrast, the corrected image 80 generated by the correction processing of the present embodiment (using the correction weight “w”) shows that signals originating from vessels in shallow layer image 60 are more effectively excluded compared to corrected image 80′. As demonstrated above, the processing of the present embodiment more effectively reduces the influence of projection artifacts in deep layer image 70.

In the present embodiment, the CPU 31 executes the following processes for each of the multiple local regions LA: calculating the shallow layer image weight “m” (S7), calculating the correction weight “w” (S8), and correcting the deep layer image 70 based on the correction weight “w” (S9). Therefore, if the influence of projection artifacts is significant in a local region LA, the correlation between shallow layer image 60 and deep layer image 70 increases, resulting in a larger correction weight “w.” On the other hand, if the influence of projection artifacts is minimal in a local region LA, the correlation between shallow layer image 60 and deep layer image 70 decreases, leading to a smaller correction weight “w.” Additionally, since the shallow layer image weight “m” is also calculated for each local region LA, the influence of outliers is appropriately reduced according to the local region LA. Thus, processing each local region LA individually enables the influence of artifacts in deep layer image 70 to be more effectively reduced.

The technology disclosed in the above embodiment is merely an example. Therefore, it is possible to modify the technology exemplified in the above embodiment. Only some of the multiple technologies exemplified in the above-described embodiment may be implemented. For example, the process of calculating the shallow layer image weight “m” (S7), the process of calculating the correction weight “w” (S8), and the process of correcting the deep layer image 70 based on the correction weight “w” (S9) may be performed on the entire image region rather than for each local region LA. Even in this case, the influence of outliers is more likely to be appropriately excluded, making it easier to effectively reduce the impact of artifacts in deep layer image 70.

The process of acquiring shallow layer image 60 and deep layer image 70 in S3 of FIG. 2 is an example of an “image acquisition step.” The process of calculating the correction weight “w” in S7 and S8 is an example of a “correction weight calculation step.” The process of correcting deep layer image 70 based on the correction weight “w” in S9 is an example of an “image correction step.”

1. An OCT image processing program executed by an OCT image processing device that processes an image acquired by an OCT device that is configured to generate motion contrast data by processing a plurality of OCT signals acquired from a same position on a living tissue at different times, the OCT image processing program, when executed by a control unit of the OCT image processing device, causing the OCT image processing device to perform:
an image acquisition step of acquiring a shallow layer image and a deep layer image which are generated based on the motion contrast data, wherein the shallow layer image is an image of a shallow region of the living tissue, and the deep layer image is an image of a deep region deeper than the shallow region;
a correction weight calculation step of calculating a correction weight for correcting the deep layer image such that a correlation between the shallow layer image and the deep layer image is reduced; and
an image correction step of correcting the deep layer image according to the calculated correction weight, wherein
at the correction weight calculation step, the correction weight is calculated using a robust estimation method.

2. The OCT image processing program according to claim 1, wherein at the correction weight calculation step, the correction weight is calculated by the robust estimation method using a shallow layer image weight calculated from the shallow layer image.

3. The OCT image processing program according to claim 1, wherein at the correction weight calculation step, the correction weight is calculated using a weighted regression analysis as the robust estimation method.

4. The OCT image processing program according to claim 3, wherein at the correction weight calculation step, when “m” is a shallow layer weight, “x” is brightness of the shallow layer image, “y” is brightness of the deep layer image, and “w” is the correction weight, then
$$w = \frac{\sum m \sum mxy - \sum mx \sum my}{\sum m \sum {mx}^{2} - {( \sum mx )}^{2}} .$$

5. The OCT image processing program according to claim 1, wherein
at the correction weight calculation step, the correction weight is calculated for each local region, and
at the image correction step, the deep layer image is corrected for each local region according to the correction weight that is calculated for each local region.

6. An OCT image processing device that processes an image acquired by an OCT device that is configured to generate motion contrast data by processing a plurality of OCT signals acquired from a same position on a living tissue at different times, the OCT image processing device comprising
a control unit configured to perform:
an image acquisition step of acquiring a shallow layer image and a deep layer image which are generated based on the motion contrast data, wherein the shallow layer image is an image of a shallow region of the living tissue, and the deep layer image is an image of a deep region deeper than the shallow region;
a correction weight calculation step of calculating a correction weight for correcting the deep layer image such that a correlation between the shallow layer image and the deep layer image is reduced; and
an image correction step of correcting the deep layer image according to the calculated correction weight, wherein
at the correction weight calculation step, the correction weight is calculated using a robust estimation method.

7. The OCT image processing device according to claim 6, wherein at the correction weight calculation step, the correction weight is calculated by the robust estimation method using a shallow layer image weight calculated from the shallow layer image.

8. The OCT image processing device according to claim 6, wherein at the correction weight calculation step, the correction weight is calculated using a weighted regression analysis as the robust estimation method.

9. The OCT image processing device according to claim 8, wherein at the correction weight calculation step, when “m” is a shallow layer weight, “x” is brightness of the shallow layer image, “y” is brightness of the deep layer image, and “w” is the correction weight, then
$$w = \frac{\sum m \sum mxy - \sum mx \sum my}{\sum m \sum {mx}^{2} - {( \sum mx )}^{2}} .$$

10. The OCT image processing device according to claim 6, wherein
at the correction weight calculation step, the correction weight is calculated for each local region, and
at the image correction step, the deep layer image is corrected for each local region according to the correction weight that is calculated for each local region.

11. An OCT image processing method for processing an image acquired by an OCT device that is configured to generate motion contrast data by processing a plurality of OCT signals acquired from a same position on a living tissue at different times, the OCT image processing method comprising:
an image acquisition step of acquiring a shallow layer image and a deep layer image which are generated based on the motion contrast data, wherein the shallow layer image is an image of a shallow region of the living tissue, and the deep layer image is an image of a deep region deeper than the shallow region;
a correction weight calculation step of calculating a correction weight for correcting the deep layer image such that a correlation between the shallow layer image and the deep layer image is reduced; and
an image correction step of correcting the deep layer image according to the calculated correction weight, wherein
at the correction weight calculation step, the correction weight is calculated using a robust estimation method.

12. The OCT image processing method according to claim 11, wherein at the correction weight calculation step, the correction weight is calculated by the robust estimation method using a shallow layer image weight calculated from the shallow layer image.

13. The OCT image processing method according to claim 11, wherein at the correction weight calculation step, the correction weight is calculated using a weighted regression analysis as the robust estimation method.

14. The OCT image processing method according to claim 13, wherein at the correction weight calculation step, when “m” is a shallow layer weight, “x” is brightness of the shallow layer image, “y” is brightness of the deep layer image, and “w” is the correction weight, then
$$w = \frac{\sum m \sum mxy - \sum mx \sum my}{\sum m \sum {mx}^{2} - {( \sum mx )}^{2}} .$$

15. The OCT image processing method according to claim 11, wherein
at the correction weight calculation step, the correction weight is calculated for each local region, and
at the image correction step, the deep layer image is corrected for each local region according to the correction weight that is calculated for each local region.
